(19) United States
(12) Patent Application Publication
(10) Pub. No.: US 2007/0073379 A1
(43) Pub. Date: Mar. 29, 2007

(54) Stent delivery system

(76) Inventor: Jean C. Chang, Saratoga, CA (US)

Correspondence Address: CARDIOMIND, INC., 257 HUMBOLDT COURT, SUNNYVALE, CA, 94089, US

(21) Appl. No.: 11/241,802

(22) Filed: Sep. 29, 2005

(51) Int. Cl. A61F 2/06

(52) U.S. Cl. 623/1.12

(57) ABSTRACT

Medical device and methods for delivery or implantation of prostheses within hollow body organs and vessels or other luminal anatomy are disclosed. The subject technologies may be used in the treatment of atherosclerosis in stenting procedures.

### BACKGROUND

Implants such as stents and occlusive coils have been used in patients for a wide variety of reasons. One of the most common “stenting” procedures is carried out in connection with the treatment of atherosclerosis, a disease that results in a narrowing and stenosis of body lumens, such as the coronary arteries. Typically, prior to stenting, an angioplasty procedure is performed to dilate the vessel at the site of the narrowing (i.e., the site of a lesion) by means of a balloon. Thereafter, a stent is set in apposition to the interior surface of the lumen at the lesion site in order to help maintain an open passageway. This result may be affected by means of scaffolding support alone or in coordinated use with one or more drugs carried by the stent to aid in preventing restenosis.

Various stent designs exist and are in use today, but self-expandable and balloon-expandable stent systems and their related deployment techniques are now predominant. Balloon-expandable stents require a mechanical force, such as exerted by a balloon disposed within the stent interior, to increase in diameter. Self-expanding stents are generally constructed of shape memory materials that are biased so that the stent diameter will increase from a reduced diameter maintained by constraining forces to an expanded diameter once the constraining forces are removed, without the action of any external mechanical forces. Because self-expanding prosthetic devices need not be set over a balloon (as with balloon-expandable designs), but may be set over the guidewire by which they are delivered (referred to as “over-the-wire”), self-expanding stent delivery systems can be designed to a relatively smaller outer diameter than their balloon-expandable counterparts. As such, self-expanding stents may be better suited to reach the smallest vasculature or achieve access in more difficult cases.

Examples of currently available self-expandable stents are the Magic WALLSTENT® stents and Radius stents (Boston Scientific). The Cypher® stent (Cordis Corporation) is a commonly used balloon-expandable stent. Additional self-expanding stent background is presented in: “An Overview of Superelastic Stent Design,” Min. Invas Ther & Allied Technol 822: 9(3/4) 235-246, “A Survey of Stent Designs,” Min. Invas Ther & Allied Technol 822: 11(4) 137-147, and “Coronary Artery Stents: Design and Biologic Considerations,” Cardiology Special Edition, 823: 9(2) 9-14, “Clinical and Angiographic Efficacy of a Self-Expanding Stent,” Am Heart J 823: 145(5) 868-874.

There are trade-offs, however, in using self-expanding stents over balloon-expandable stents. Because self-expanding stents are biased to expand, a retention means, often one that surrounds the stent, is used. A shortcoming of delivery systems for self-expanding stents using a delivery catheter which provides an outer sheath for retaining the stent prior to deployment is that they tend to have larger profiles and be less flexible than delivery systems for balloon-expandable stents. As such, prior art self-expanding stent delivery systems are limited by the inability to navigate tortuous and narrow passageways.

Examples of self-expanding stent deployment systems are presented in U.S. Pat. No. 4,830,003 (Wolff, et al.) and U.S. Pat. No. 5,064,435 (Porter). In each, an outer sheath restraining a stent overrides an inner tubular member. The tubular member has a lumen adapted to receive a guidewire and a distal end adapted to abut the stent for delivery. Another such delivery system is described in U.S. Pat. No. 4,580,568 (Gianturco) in which a sheath overrides a polymeric tubular member. U.S. Pat. No. 6,280,465 (Cryer) discloses a very similar system. The device described in connection with FIG. 4 of Cryer includes a central guidewire member, over which a tubular sheath and pusher are disposed. In use, the guidewire/pusher/sheath combination is advanced to a treatment site within a guiding catheter as an integral assembly. The ability to mount the stent and its retention means to any guidewire is expressed as desirable. Unit preassembly is also discussed as advantageous for time savings.

Irrespective of their various asserted advantages, all of these known sheath/pusher systems are limited in the degree to which the systems can be miniaturized. Limiting factors include the fact that the pusher must have sufficient wall thickness to offer an adequate interface to abut the stent and that additional clearance space between the elements must be provided as sufficient to allow relative movement between the pusher, sheath and guidewire.

The latter issue is addressed in U.S. Patent Application Publication No. 2003/0163156 (Hebert, et al.). This device integrates stent pusher and guidewire functions. The embodiment of FIG. 3 of the Hebert application provides a guidewire having a reduced, constant diameter section upon which the stent is mounted. The mounting region is straddled by tapered diameter sections situated proximally and distally of the stent, where the tapered distal section has diameters which are smaller than those of the tapered proximal section. The embodiment of FIG. 4 of the Hebert application takes a different approach in which a uniform diameter guide wire has a stepped-down, constant diameter section upon which the stent is mounted. The most distal section of the guidewire then has a tapered section.

Both embodiments have their shortcomings. The reduced proximal diameter of the embodiment of FIG. 3 enhances flexibility of the guidewire, but Applicant has observed that leaving unoccupied space between a central member and outer sheath negatively impacts delivery system deployment performance, possibly resulting in unintended advancement of the tip of the delivery system (together with the stent) upon sheath withdrawal. On the other hand, the embodiment of FIG. 4, while alleviating this concern, is less flexible.

Accordingly, a need for stent space-efficient delivery systems with improved performance persists. It would be additionally beneficial-to provide such a delivery system which, as a whole, functions as a lead wire in which the guidewire is multi-functional above and beyond functioning solely in delivery of the stent. In particular, it would be advantageous to provide such a guidewire which functions as a guidewire for other delivery system, e.g., an angioplasty balloon, or provides additional features unrelated to delivery, e.g., embolic protection, where such functions and features may be employed either prior to or after stent delivery or deployment.

The present invention offers such functions and features, but in a higher performance package able to access and deliver one or more stents to sites including the neurovasculature, especially within the brain, and small vessels, particularly distal coronary arteries.

### SUMMARY OF THE INVENTION

In accordance with the present invention, a delivery system is provided for use in deliverying an implantable device to within the body. The subject systems are particularly useful for delivery and deploying a stent within the vasculature. In certain embodiments, the device can be used as a lead guidewire for the delivery of other systems or components (e.g., angioplasty balloons, embolic filters, etc.), either subsequent to or prior to delivery or deployment of the stent.

The subject delivery systems include an outer sleeve or tubular sheath to restrain one or more stents carried on the distal end of an inner member. The inner member comprises a corewire, over which a stent is releasably mounted to the distal portion of the corewire. At the very distal end of the corewire is an optional coil tip to facilitate translation of the system in tortuous or otherwise difficult to access anatomy. The inner member further comprises a cladding layer or covering, preferably having a low modulus of elasticity (i.e., at least as low as that of the corewire so as not to limit flexibility of the corewire), bonded to the corewire up to a location just proximal of the section of the corewire to which the stent is to be positioned.

As such, the distal end of the cladding may serve as a stent stop, blocker or abutment interface. Alternatively, a separate stop component may be mounted to the distal end of the cladding.

Having a substantially constant outer diameter, the cladding serves to fill space between the guidewire core and the outer sheath. Employing a cladding material as opposed to increased (constant) diameter core member offers a number of advantages as elaborated upon below. Moreover, the inventive corewire/cladding combination, i.e., the inner member, is configured to maximize the often conflicting objectives of flexibility and pushability.

One manner in which flexibility is achieved is by varying the diameter of the corewire, particularly a distal portion of the corewire. With certain variations of the invention, the corewire is tapered from a larger diameter at a more proximal end to a smaller diameter at a distal end. The “taper” may be a continuous taper or a varied taper involving a step-down in size over sections. The tapered portions of the corewire are complimented by substantially corresponding reversely tapered portions within the cladding. As such, the inner member comprises a substantially constant outer diameter body. This aspect allows for closer tolerance between the outer sleeve and the inner member. Such a system may offer each of better pushability, trackability and implant deployment characteristics. Yet, the cladding material does not inhibit the flexibility of the tapered corewire. Indeed, its properties can be tuned to complement those of the corewire.

Another way in which the various advantages of flexibility, pushability and improved delivery performance may be achieved is by employing a plurality of cladding sections of different modulus over the corewire. In one example, a constant outer diameter cladding (comprising one or more pieces) is bonded to a constant diameter corewire where the modulus of elasticity of the cladding is reduced proximally-to-distally along at least a portion of its length. By selectively adjusting the composition and/or length of sections of cladding material along the length of at least a distal portion of the delivery system, performance of the inner member may be optimized in terms of flexibility as well as the pushability while maintaining a substantially constant outer diameter.

In certain embodiments, the corewire provides additional functions or carries components in addition to the stent. Specifically, the corewire may provide a filter device (e.g., an embolic filter) which is usable prior to (e.g., during an angioplasty procedure), during and/or after stent deployment. The corewire may further include radiopaque markers at selected locations along its distal length to demark, for example, the very distal tip, the filter location and/or the stent location.

The present invention provides a delivery system that may have an outer distal (sheath/sleeve) diameter of about 2 Fr (about 0.022 to 0.026 inch) or less and is adapted to deliver elastic/superelastic self-expanding stents. Specifically, the delivery system may have a crossing profile of

Methodology described in association with the systems and devices disclosed also forms part of the invention. Such methodology may include that associated with completing an angioplasty, bridging an aneurysm, deploying radially-expandable anchors for pacing leads or an embolic filter, or placement of a prosthesis within neurovasculature, an organ selected from the kidney and liver, within reproductive anatomy such as selected vasdeferens and fallopian tubes or other applications.

These and other objects, advantages, and features of the invention will become apparent to those persons skilled in the art upon reading the details of the invention as more fully described below.

### DEFINITIONS

The term “stent” as used herein includes any stent, such as coronary artery stents, other vascular prosthesis, or other radially expanding or expandable prosthesis or scaffold-type implant suitable for the noted treatments or otherwise. Exemplary structures include wire mesh or lattice patterns and coils, though others may be employed in the present invention.

A “self-expanding” stent as used herein is a scaffold-type structure (serving any of a number of purposes) that expands from a reduced-diameter (be it circular or otherwise) configuration to an increased-diameter configuration. The mechanism for shape recover may be elastic or pseudoelastic. While it is generally desireable to employ an alloy (such as nickel-titanium, or Nitinol alloy) set for use as a superelastic alloy, it may alternatively employ thermal shape memory properties to drive expansion upon release.

A “wire” as used herein generally comprises a common metallic member. However, the wire may be coated or covered by a polymeric material (e.g., with a lubricious material such as TEFLON®, i.e., PolyTetraFluoroEthelyne or PTFE) or otherwise. Still further, the “wire” may be a hybrid structure with metal and a polymeric material (e.g., Vectran™, Spectra™, Nylon, etc.) or composite material (e.g., carbon fiber in a polymer matrix). The wire may be a filament, bundle of filaments, cable, ribbon or in some other form. It is generally not hollow.

A “corewire” or “core member” may be use interchangeably and, as referred to herein, has a wire form and may be made from any biocompatible material including; but not limited to, stainless steel and any of its alloys; titanium alloys, e.g., Ni—Ti alloys; other shape memory alloys (i.e., SMAs); tantalum; polymers, e.g., polyethylene and copolymers thereof, polyethylene terephthalate or copolymers thereof, nylon, silicone, polyurethane fluoropolymers, poly(vinylchloride), and combinations thereof.

An “inner member” as disclosed herein includes a core member or a corewire and a cladding, cladding sections or a cladding layer which covers or surrounds at least a portion of the core member or corewire. The two may be bonded together or otherwise connected/interconnected.

A “cladding” as referred to herein means an outer layer of material which is bonded to a core member or a core wire. As with the “wire” discussed above, the material defining the cladding may be metallic, polymeric or a hybrid of thereof or a composite material. The cladding material may have the same flexibility or greater flexibility than the member to which it is bonded to so as not impeded the member's flexibility.

A “hypotube” or “hypotubing” as referred to herein means small diameter tubing in the size range discussed below, generally with a thin wall. The hypotube may specifically be hypodermic needle tubing. Alternatively, it maybe wound or braided cable tubing, such as provided by Asahi Intec Co., Ltd. or otherwise. As with the “wire” discussed above, the material defining the hypotube may be metallic, polymeric or a hybrid of metallic and polymeric or composite material.

A “sleeve” as referred to herein may be made of hypotubing or otherwise constructed. The sleeve may be a tubular member, or it may have longitudinal opening(s). It is an outer member, able to slidingly receive and hold at least a portion of an inner member.

An “atraumatic tip” may comprise a plurality of spring coils attached to a tapered wire section. At a distal end of the coils typically terminate with a bulb or ball that is often made of solder. In such a construction, the coils and/or solder are often platinum alloy or another radiopaque material. The coils may also be platinum, or be of another material. In the present invention, the wire section to which the coils are attached may be tapered, but need not be tapered. In addition, alternate structures are possible. In one example, the atraumatic tip may comprise a molded tantalum-loaded 35 durometer Pebax™ tip. However constructed, the atraumatic tip may be straight or curved, the latter configuration possibly assisting in directing or steering the delivery guide to a desired intravascular location.

To “connect” or to have or make a “connection” between parts refers to fusing, bonding, welding (by resistance, laser, chemically, ultrasonically, etc.), gluing, pinning, crimping, clamping or otherwise mechanically or physically joining, attaching or holding components together (permanently or temporarily).

To “bond” or form a “bonding” between structures refers to forming an intimate contact between the structures, typically where the contanct is intended to be permanent. The bond or bonding may be achieved by any known means and process including, but not limited to, pressure rolling, extruding, drawing, swaging and adhesion.

“Radiopaque markers” are understood to be markers or features of the various delivery system components, corewire or implant that may be employed to facilitate visualization of the system components. As such, various platinum (or other radiopaque material) bands or other markers (such as tantalum plugs) may be variously incorporated into the system. Alternatively, or additionally, the stent may be made of radiopaque material or incorporate them. Especially where the stent employed may shorten somewhat upon deployment, it may also be desired to align radiopaque features with the expected location (relative to the body of the inner member) of the stent upon deployment. A filter used with the subject devices may also be made of radiopaque material for the same reasons.

### BRIEF DESCRIPTIONS OF THE DRAWINGS

The figures shown herein are not necessarily drawn to scale, with some components and features being exaggerated for clarity. Each of the figures diagrammatically illustrates aspects of the invention. Of these:

FIG. 1 shows a heart in which its vessels may be the subject of one or more angioplasty and stenting procedures;

FIG. 2A shows an expanded stent cut pattern as may be used in producing a stent according to a first aspect of the invention; FIG. 2B shows a stent cut pattern for a second stent produced according to another aspect of the present invention;

FIG. 3A shows an expanded stent cut pattern as may be used in producing a stent according to a first aspect of the invention; FIG. 3B shows a stent cut pattern for a second stent produced according to another aspect of the present invention;

FIGS. 4A-4L illustrate stent deployment methodology to be carried out with the subject delivery guide member;

FIG. 5 provides an overview of a delivery system incorporating a tubular member according to the present invention;

FIG. 6 shows an exemplary variation of a subject delivery system having a cladding-covered corewire;

FIG. 7 shows another exemplary variation of a subject delivery system having a corewire provided with an embolic filter; and

FIG. 8 shows an exemplary variation of a subject delivery system having a cladding-covered corewire provided with an embolic filter.

Variation of the invention from the embodiments pictured is, of course, contemplated.

### DETAILED DESCRIPTION OF THE INVENTION

Various exemplary embodiments of the invention are described below. Reference is made to these examples in a non-limiting sense. They are provided to illustrate more broadly applicable aspects of the present invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. All such modifications are intended to be within the scope of the claims made herein.

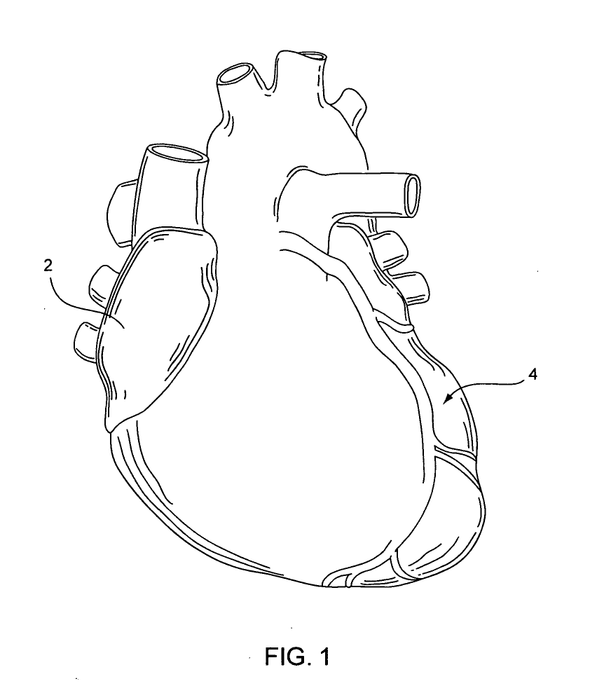

In light of this framework, FIG. 1 shows a heart 2 in which its vessels may be the subject of one or more angioplasty and/or stenting procedures. To date, however, significant difficulty or impossibility is confronted in reaching smaller coronary arteries 4. If a stent and a delivery system could be provided for accessing such small vessels and other difficult anatomy, an additional 20 to 25% of coronary percutaneous procedures could be performed with such a system. Such potential offers opportunity for huge gains in human healthcare and a concomitant market opportunity in the realm of roughly $1 to 2 billion U.S. dollars—with the further benefit of avoiding loss of income and productivity of those treated.

Features of the present invention are uniquely suited for a system able to reach small vessels (though use of the subject systems s not limited to such a setting.) By “small” coronary vessels, it is meant vessels having a inside diameter between about 1.5 or 2 and about 3 mm in diameter. These vessels include, but are not limited to, the Posterior Descending Artery (PDA), Obtuse Marginal (OM) and small diagonals. Conditions such as diffuse stenosis and diabetes produce conditions that represent other access and delivery challenges which can be addressed with a delivery system according to the present invention. Other extended treatment areas addressable with the subject systems include vessel bifurcations,-chronic total occlusions (CTOs), and prevention procedures (such as in stenting of vulnerable plaque).

Assuming a means of delivering one or more appropriately-sized stents, it may be preferred to use a drug eluting stent (DES) in such an application to aid in preventing restenosis. A review of suitable drug coatings and available vendors is presented in “DES Overview: Agents, release mechanism, and stent platform” a presentation by Campbell Rogers, MD incorporated by reference in its entirety. However, bare-metal stents may be employed in the present invention.

Yet, with an appropriate deployment system, self-expanding stents may offer one or more of the following advantages over balloon-expandable models: 1) greater accessibility to distal, tortuous and small vessel anatomy—by virtue of decreasing crossing diameter and increasing compliance relative to a system requiring a deployment balloon, 2) sequentially controlled or “gentle” device deployment, 3) use with low pressure balloon pre-dilatation (if desirable) to reduce barotraumas, 4) strut thickness reduction in some cases reducing the amount of “foreign body” material in a vessel or other body conduit, 5) opportunity to treat neurovasculature—due to smaller crossing diameters and/or gentle delivery options, 6) the ability to easily scale-up a successful treatment system to treat larger vessels or vice versa, 7) a decrease in system complexity, offering potential advantages both in terms of reliability and system cost, 8) reducing intimal hyperplasia, and 9) conforming to tapering anatomy—without imparting complimentary geometry to the stent (though this option exists as well).

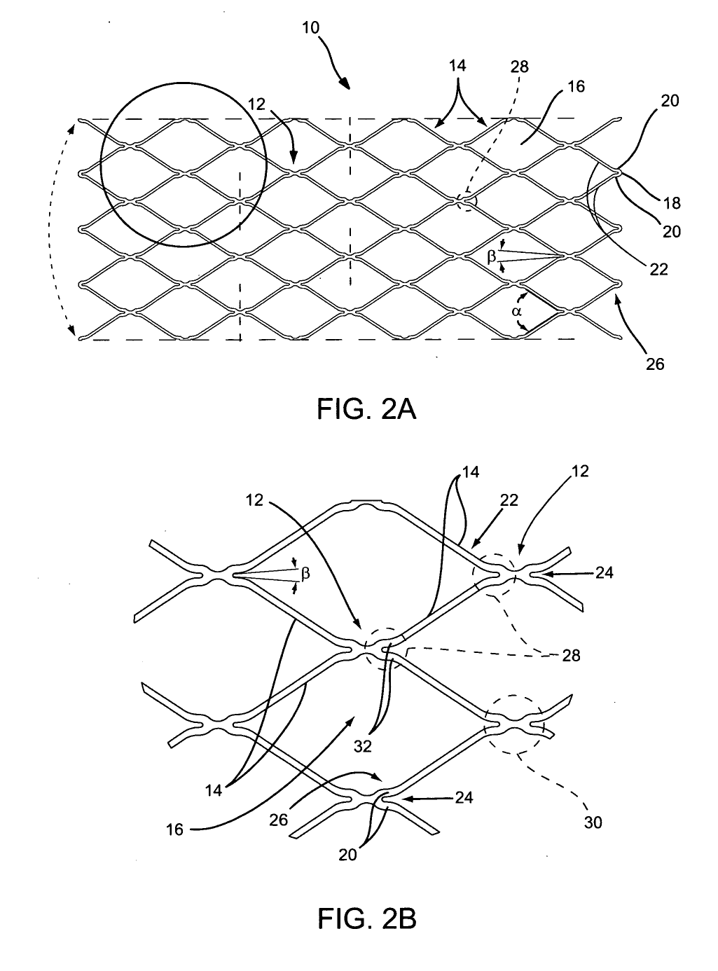

At least some of these noted advantages may be realized using a stent 10 as shown in FIG. 2A. The stent pattern pictured is well suited for use in small vessels. It may be collapsed to an outer diameter of about 0.018 inch (0.46 mm), or even smaller to about 0.014 inch (0.36 mm)—including the restraint/joint used to hold it down—and expanded to a size (fully unrestrained) between about 1.5 mm (0.059 inch) or 2 mm (0.079 inch) or 3 mm (0.12 inch) and about 3.5 mm (0.14 inch).

In use, the stent will be sized so that it is not fully expanded when fully deployed against the wall of a vessel in order to provide a measure of radial force thereto (i.e., the stent will be “oversized” as discussed above). The force will secure the stent and offer potential benefits in reducing intimal hyperplasia and vessel collapse or even pinning dissected tissue in apposition.

Stent 10 preferably comprises NiTi that is superelastic at about room temperature (i.e., as in having an Af as low as 15° C. or even 0° C.). Also, the stent is preferably electropolished to improve biocompatibility and corrosion and fatigue resistance. The stent may be a DES unit. The drug can be directly applied to the stent surface(s), or introduced into pockets or an appropriate matrix set over at least an outer portion of the stent. The stent may be coated with gold and/or platinum to provide improved radiopacity for viewing under medical imaging.

For a stent able to collapse to an outer diameter of about 0.012 inches and expand to about 3.5 mm, the thickness of the NiTi is about 0.0025 inch (0.64 mm). Such a stent is designed for use in a 3 mm vessel or other body conduit, thereby providing the desired radial force in the manner noted above. Further information regarding radial force parameters in coronary stents may be noted in the article, “Radial Force of Coronary Stents: A Comparative Analysis,” Catheterization and Cardiovascular Interventions 46: 380-391 (1999), incorporated by reference herein in its entirety.

In one manner of production, the stent in FIG. 2A is laser or EDM cut from round NiTi tubing, with the flattened-out pattern shown wrapping around the tube as indicated by dashed lines. In such a procedure, the stent is preferably cut in its fully-expanded shape. By initially producing the stent to full size, the approach allows cutting finer details in comparison to simply cutting a smaller tube with slits and then heat-expanding/annealing it into its final (working) diameter. Avoiding post-cutting heat forming also reduces production cost as well as the above-reference effects.

Regarding the finer details of the subject stent, as readily observed in the detail view provided in FIG. 2B, necked down bridge sections 12 are provided between axially/horizontally adjacent struts or arms/legs 14, wherein the struts define a lattice of closed cells 16. Terminal ends 18 of the cells are preferably rounded-off so as to be atraumatic.

To increase stent conformability to tortuous anatomy, the bridge sections can be strategically separated or opened as indicated by the broken lines in FIG. 2A. To facilitate such tuning of the stent, the bridge sections are sufficiently long so that fully rounded ends 18 may be formed internally to the lattice just as shown on the outside of the stent if the connection(s) is/are severed to separate adjacent cells 16. Whether provided as ends 18 or adjoined by a bridge section 12, junction sections 28 connect circumferentially or vertically adjacent struts (as illustrated). Where no bridge sections are provided, the junction sections can be unified between horizontally adjacent stent struts as indicated in region 30.

The advantage of the optional double-concave profile of each strut bridge 12 is that it reduces material width (relative to what would otherwise be presented by a parallel side profile) to improve flexibility and thus trackability and conformability of the stent within the subject anatomy while still maintaining the option for separating/breaking the cells apart.

Further optional features of stent 10 are employed in the cell end regions 18 of the design. Specifically, strut ends 20 increase in width relative to medial strut portions 22. Such a configuration distributes bending (during collapse of the stent) preferentially toward the mid region of the struts. For a given stent diameter and deflection, longer struts allow for lower stresses within the stent (and, hence, a possibility of higher compression ratios). Shorter struts allow for greater radial force (and concomitant resistance to a radially applied load) upon deployment.

In order to increase stent compliance so that it collapses as much as possible, accommodation is made for the stiffer strut ends 20 provided in the design shown in FIG. 2A. Namely, the gap 24 between the strut ends 22 is set at a smaller angle as if the stent were already partially collapsed in that area. Thus, the smaller amount of angular deflection that occurs at ends 20 can bring the sections parallel (or nearly so) when the strut medial portions 22 are so-arranged. In the variation of the invention in FIG. 2A, radiused or curved sections 26 provide a transition from a medial strut angle α (ranging from about 85 degrees to about 60 degrees) to an end strut angle β (ranging from about 30 to about 0 degrees) at the strut junctions 28 and/or extensions therefrom.

In addition, it is noted that gap 24 an angle β may actually be configured to completely close prior to fully collapsing angle α. The stent shown is not so-configured. Still, the value of doing so would-be to limit the strains (and hence, stresses) at the strut ends 22 and cell end regions 18 by providing a physical stop to prevent further strain.

In the detail view of FIG. 2B, angle β is set at 0 degrees. The gap 24 defined thereby by virtue of the noticeably thicker end sections 20 at the junction result in very little flexure along those lever arms. The strut medial portions are especially intended to accommodate bending. In addition, a hinging effect at the corner or turn 32 of junction section 28 may allow the strut to swing around angle α to provide the primary mode for compression of the stent.

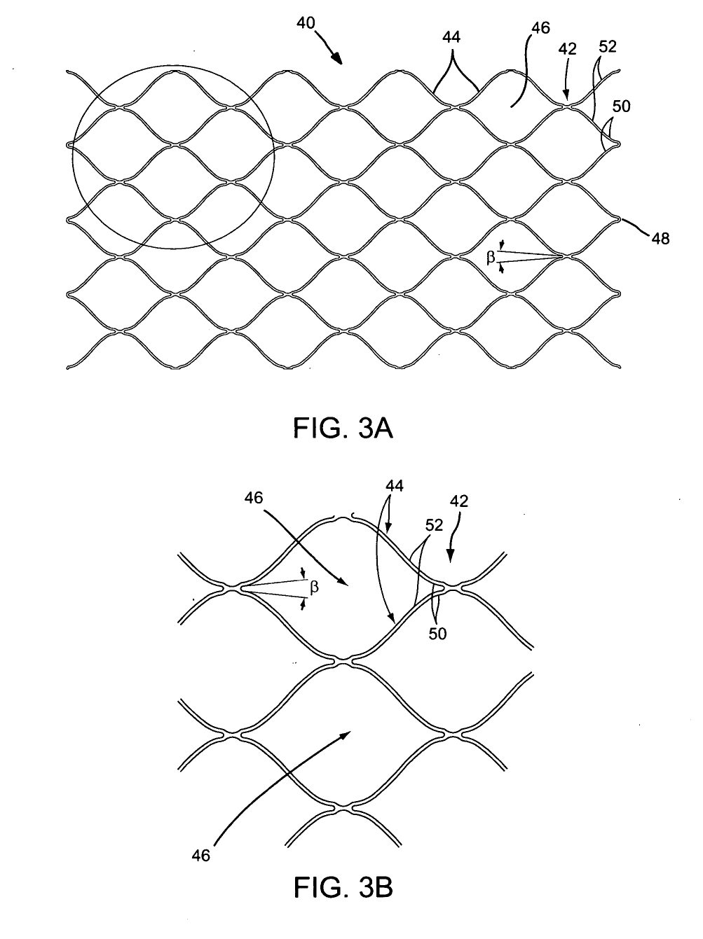

The stent pattern shown in FIG. 3A and detailed in FIG. 3B offers certain similarities as well as some major differences from the stent pattern presented in FIGS. 2A and 2B. As in the variation above, stent 40 includes necked down bridge sections 42 provided between adjacent struts or arms/legs 44, wherein the struts define a lattice of closed cells 46. In addition, terminal ends 48 of the cells are preferably rounded-off so as to be atraumatic.

Furthermore, the bridge sections 42 of stent 40 can be separated for compliance purposes. In addition, they may be otherwise modified (e.g., as described above) or even eliminated. Also, in each design, the overall dimensions of the cells and indeed the number of cells provided to define axial length and/or diameter may be varied (as indicated by the vertical and horizontal section lines in FIG. 3A).

Like the previous stent design, strut ends 50 may offer some increase in width relative to medial strut portions 52. However, as shown in FIG. 3B, as compared to FIG. 2B, the angle β is relatively larger. Such a configuration is not concerned with developing a hinge section and a relatively stiffer outer strut section. Instead, angle β in the FIG. 3A/3B design is meant to collapse and the strut ends are meant to bend in concert with the medial strut portions so as to essentially straighten-out upon collapsing the stent, generally forming tear-drop spaces between adjacent struts. This approach offers a stress-reducing radius of curvature where struts join, and maximum stent compression.

The “S” curves defined by the struts are produced in a stent cut to a final or near final size (as shown in FIGS. 3A and 3B). The curves are preferably determined by virtue of their origination in a physical or computer model that is expanded from a desired compressed shape to the final expanded shape. So derived, the stent can be compressed or collapsed under force to provide an outer surface profile that is as solid or smooth and/or cylindrical as possible or feasible.

Such action is enabled by distribution of the stresses associated with compression to generate stains to produce the intended compressed and expanded shapes. This effect is accomplished in a design unaffected by one or more expansion and heat setting cycles that otherwise deteriorate the quality of the superelastic NiTi stent material. Further details regarding the “S” stent design and alternative stent constructions as may be used in the present invention are disclosed in U.S. Provisional Patent Application Ser. No. 60/619,437, entitled, “Small Vessel Stent Designs”, filed Oct. 14, 2004 and incorporated herein by reference in its entirety. In the case of each of the above stent designs, by utilizing a stent design that minimizes problematic strain (and in the latter case actually uses the same to provide an improved compressed profile), very high compression ratios of the stent may be achieved from about 5× to about 10× or above.

Delivery systems according to the present invention are advantageously sized to correspond to existing guidewire sizes. For example, the system may have about an 0.014 (0.36 mm), 0.018 (0.46 mm), 0.022 (0.56 mm), 0.025 (0.64 mm) inch crossing profile. Of course, intermediate sizes may be employed as well, especially for full-custom systems. Still further, it is contemplated that the system sizing may be set to correspond to French (FR) sizing. In that case, system sizes contemplated range at least from about 1 to about 2 FR, whereas the smallest known balloon-expandable stent delivery systems are in the size range of about 3 to about 4 FR. In instances where the overall device crossing profile matches a known guidewire size, they may be used with off-the-shelf components such as balloon and microcatheters.

At least when produced in the smallest sizes (whether in an even/standard guidewire or FR size, or otherwise), the system enables a substantially new mode of stent deployment in which delivery is achieved through an angioplasty balloon catheter or small microcatheter lumen. Further discussion and details of “through the lumen” delivery is presented in U.S. patent application Ser. No. 10/746,455 “Balloon Catheter Lumen Based Stent Delivery Systems” filed on Dec. 24, 2003 and its PCT counterpart US2004/008909 filed on Mar. 23, 2004, each incorporated by reference in its entirety.

In larger sizes, (i.e., up to about 0.035 inch crossing profile or more), the system is most applicable to peripheral vessel applications as elaborated upon below. Yet, even in “small vessel” cases or applications (where the vessel to be treated has a diameter up to about 3.0 mm), it may also be advantageous to employ a stent delivery system sized at between about 0.022 to about 0.025 inch in diameter. Such a system can be used with catheters compatible with 0.022 and/or 0.025 inch diameter guidewires.

While such a system may not be suitable for reaching the very smallest vessels, this variation of the invention is quite advantageous in comparison to known systems in reaching the larger of the small vessels (i.e., those having a diameter of about 2.5 mm or larger). By way of comparison, among the smallest known over-the-guidewire delivery systems are the Micro-Driver™ by Medtronic and Pixel™ systems by Guidant. These are adapted to treat vessels between 2 and 2.75 mm, the latter system having a crossing profile of 0.036 inches (0.91 mm). A system described in U.S. Patent Publication No. 2002/0147491 for treating small vessels is supposedly capable of downsizing to 0.026 inch (0.66 mm) in diameter. Furthermore, because the core member of the subject device can be used as a guidewire (in one fashion or another) after stent delivery, the present invention offers further advantages in use as elaborated upon below.

As referenced above, it may be desired to design a variation of the subject system for use in deploying stents in larger, peripheral vessels, biliary ducts or other hollow body organs. Such applications involve a stent being emplaced in a region having a diameter from about 3.5 to 13 mm (0.5 inch). In which case, a 0.035 to 0.039 inch (3 FR) diameter crossing profile system is advantageously provided in which the stent expands (unconstrained) to a size between about roughly 0.5 mm and about 1.0 mm greater than the vessel or hollow body organ to be treated. Sufficient stent expansion is easily achieved with the exemplary stent patterns shown in FIG. 2A/2B or 3A/3B.

Again, as a matter of comparison, the smallest delivery systems known to applicants for stent delivery in treating such larger-diameter vessels or biliary ducts is a 6 FR system (nominal 0.084 inch outer diameter), which is suited for use in an 8 FR guiding catheter. Thus, even in the larger sizes, the present invention affords opportunities not heretofore possible in achieving delivery systems in the size range of a commonly used guidewire, with the concomitant advantages discussed herein.

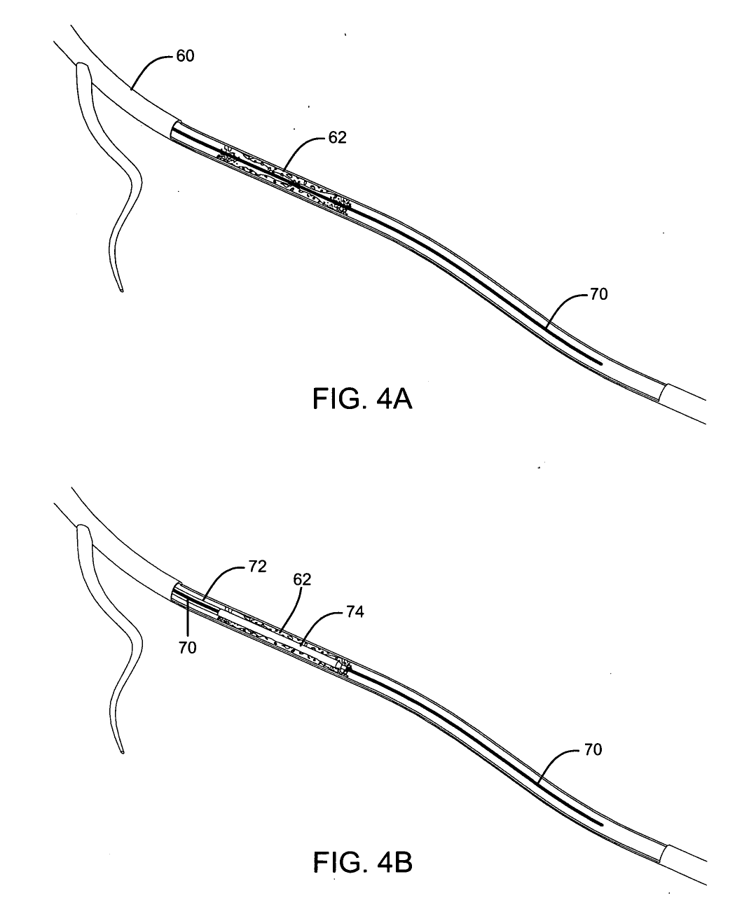

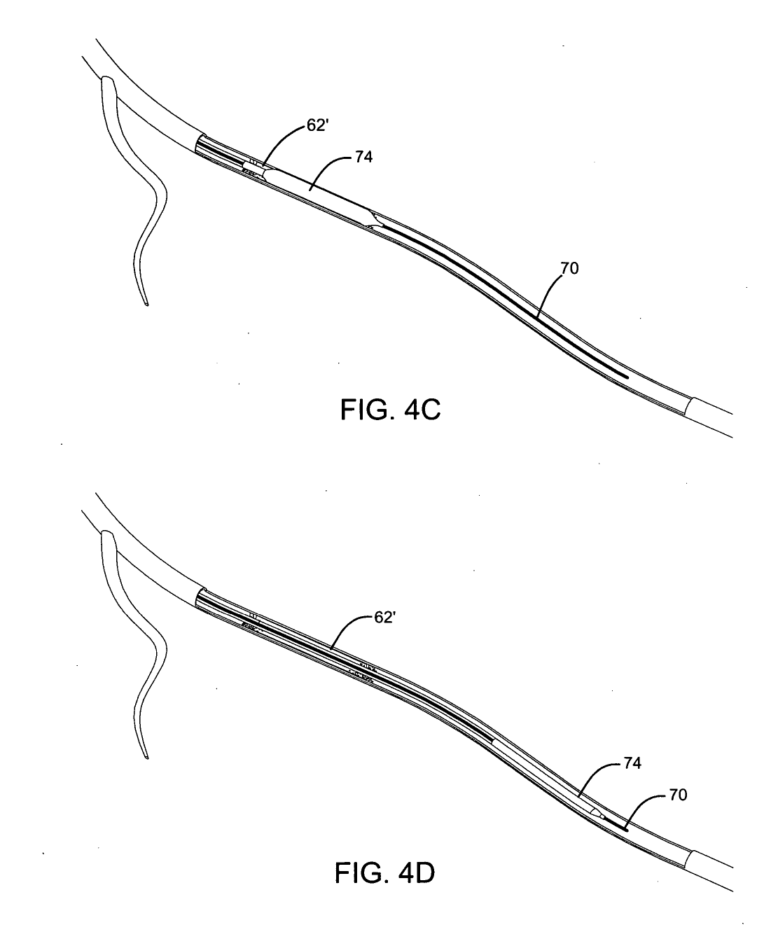

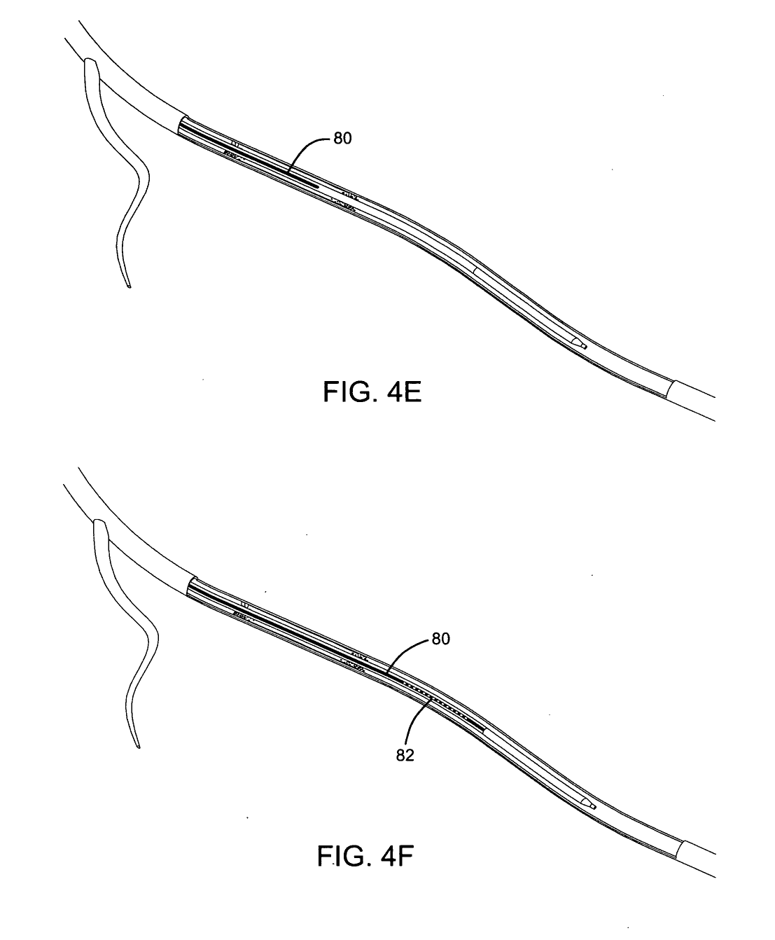

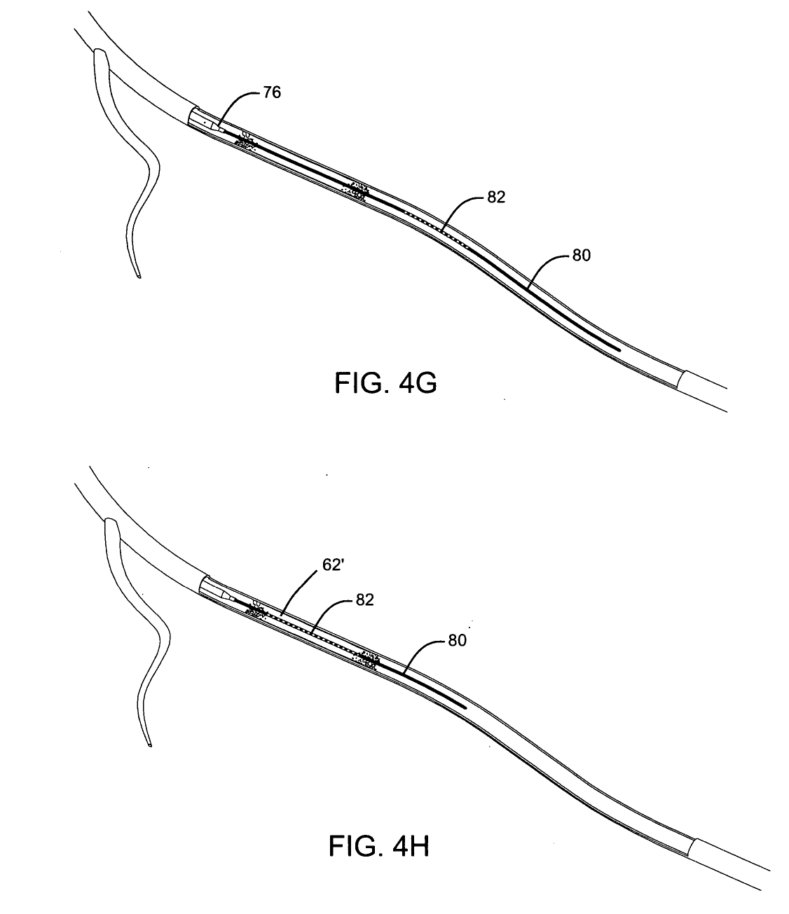

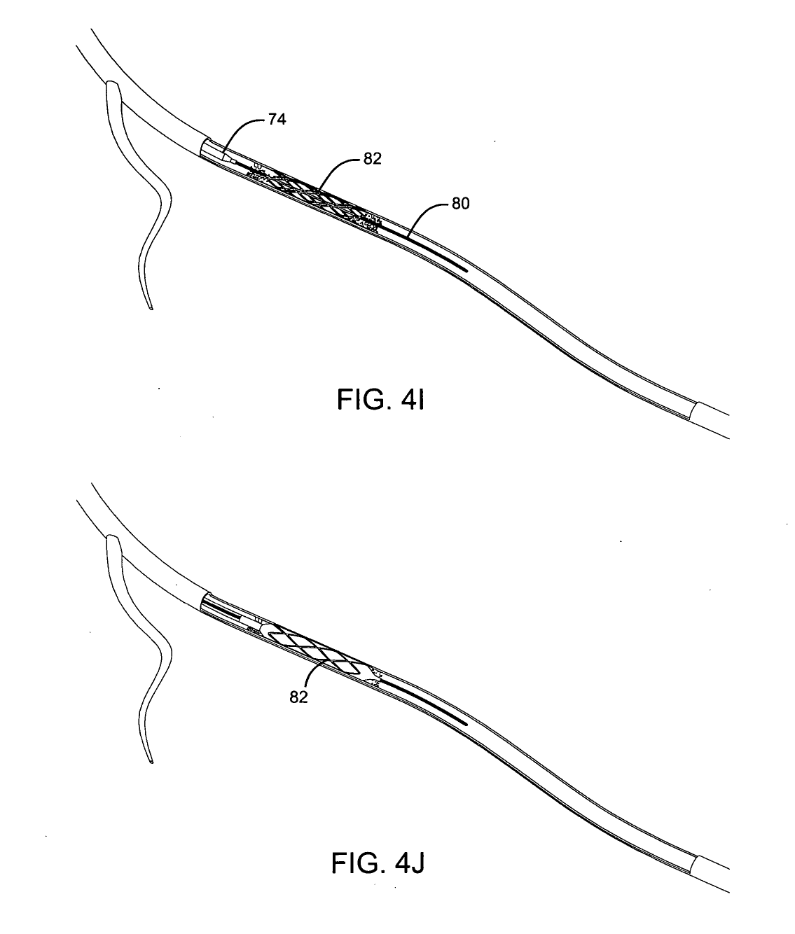

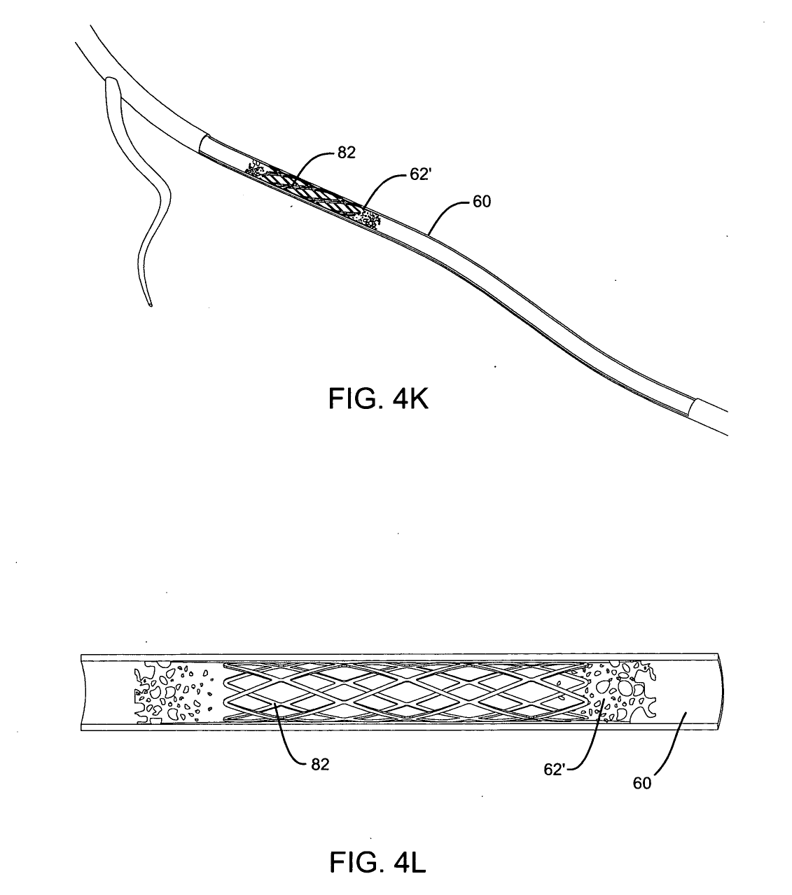

As for the manner of using the inventive system as optionally configured, FIGS. 4A-4L illustrate an exemplary angioplasty procedure. Still, the delivery systems and stents or implants described herein may be used otherwise—especially as specifically referenced herein.

Turning to FIG. 4A, it shows a coronary artery 60 that is partially or totally occluded by plaque at a treatment site/lesion 62. Into this vessel, a guidewire 70 is passed distal to the treatment site. In FIG. 4B, a balloon catheter 72 with a balloon tip 74 is passed over the guidewire, aligning the balloon portion with the lesion (the balloon catheter shaft proximal to the balloon is shown in cross section with guidewire 70 therein).

As illustrated in FIG. 4C, balloon 74 is expanded (dilatated or dilated) in performing an angioplasty procedure, opening the vessel in the region of lesion 62. The balloon expansion may be regarded as “predilatation” in the sense that it will be followed by stent placement (and optionally) a “postdilataton” balloon expansion procedure.

Next, the balloon is at least partially deflated and passed forward, beyond the dilate segment 62′ as shown in FIG. 4D. At this point, guidewire 70 is removed as illustrated in FIG. 4E. It is exchanged for a delivery guide member 80 carrying stent 82 as further described below. This exchange is illustrated in FIGS. 4E and 4F.

However, it should be appreciated that such an exchange need not occur. Rather, the original guidewire device inside the balloon catheter (or any other catheter used) may be that of item 80 (i.e., the delivery system), instead of the standard guidewire 70 shown in FIG. 4A. Thus, the steps depicted in FIGS. 4E and 4F (hence, the figures also) may be omitted. In addition, there may be no use in performing the step in FIG. 4D of advancing the balloon catheter past the lesion, since such placement is merely for the purpose of avoiding disturbing the site of the lesion by moving a guidewire past the same.

FIG. 4G illustrates the next act in either case. Particularly, the balloon catheter is withdrawn so that its distal end 86 clears the lesion. Preferably, delivery guide 80 is held stationary, in a stable position. After the balloon is pulled back, so is delivery device 80, positioning stent 82 where desired. Note, however, that simultaneous retraction may be undertaken, combining the acts depicted in FIGS. 4G and 4H. Whatever the case, it should also be appreciated that the coordinated movement will typically be achieved by virtue of skilled manipulation by a doctor viewing one or more radiopaque features associated with the stent or delivery system under medical imaging.

Once placement of the stent across from dilated segment 62′ is accomplished, stent deployment commences. The manner of deployment is elaborated upon below. Upon deployment, stent 82 assumes an at least partially expanded shape in apposition to the compressed plaque as shown in FIG. 41. Next, the aforementioned predilatation may be effected as shown in FIG. 4J by positioning balloon 74 within stent 82 and expanding both. This procedure may further expand the stent, pushing it into adjacent plaque—helping to secure each.

Naturally, the balloon need not be reintroduced for postdilatation, but it may be preferred. Regardless, once the delivery device 80 and balloon catheter 72 are withdrawn as in FIG. 4K, the angioplasty and stenting procedure at the lesion in vessel 80 is complete. FIG. 4L shows a detailed view of the emplaced stent and the desired resultant product in the form of a supported, open vessel.

In the above description, a 300 cm extendable delivery system is envisioned. Alternatively, the system can be 190 cm to accommodate a rapid exchange of monorail type of balloon catheter as is commonly known in the art. Of course, other approaches may be employed as well.

Furthermore, other endpoints may be desired such as implanting and anchoring stent in a hollow tubular body organ, closing off an aneurysm, delivering a plurality of stents, etc. In performing any of a variety of these or other procedures, suitable modification will be made in the subject methodology. The procedure shown is depicted merely because it illustrates a preferred mode of practicing the subject invention, despite its potential for broader applicability.

Furthermore, it is to be recognized that the subject invention may be practiced to perform “direct stenting.” That is, a stent may be delivered alone to maintain a body conduit, without preceding balloon angioplasty. Likewise, once one or more stents are delivered with the subject system (either by a single system, or by using multiple systems) the post-dilatation procedure(s) discussed above are merely optional. In addition, other endpoints may be desired such as implanting an anchoring stent in a hollow tubular body organ, closing off an aneurysm, delivering a plurality of stents, etc. In performing any of a variety of these or other procedures, suitable modification will be made in the subject methodology. The procedure shown is depicted merely because it illustrates a preferred mode of practicing the subject invention, despite its potential for broader applicability.

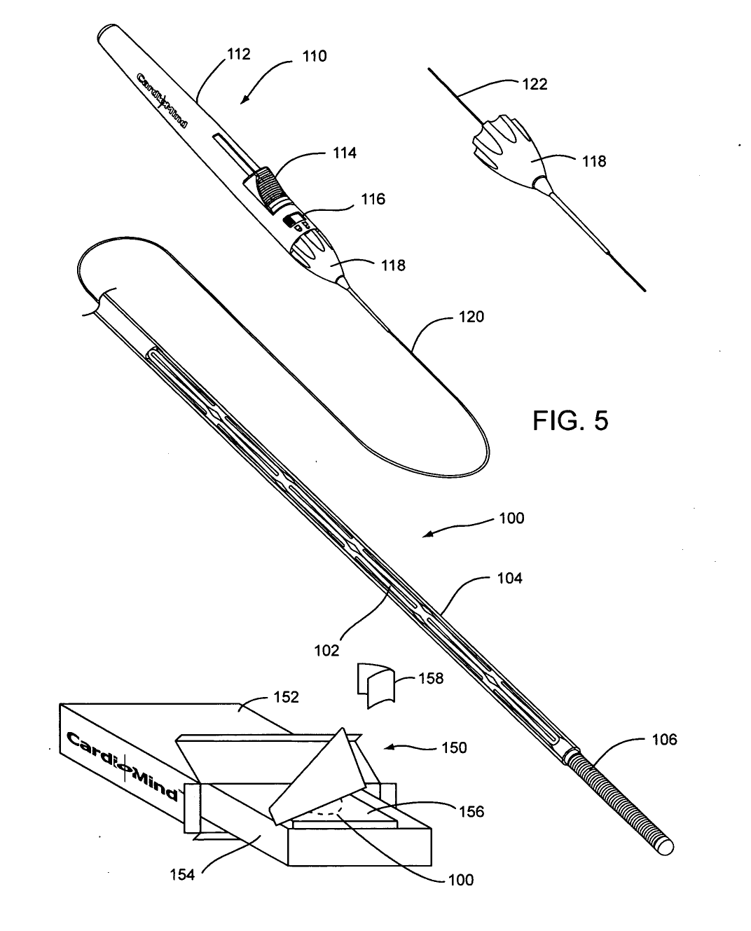

A more detailed view of the subject delivery systems is provided in FIG. 5. Here, a delivery system 100 is shown along with a stent 102 shown in a collapsed configuration upon the delivery guide member. A tubular restraint assembly 104 is provided over and around the stent to restrain it from expanding.

Irrespective of the restraint approach selected, the proximal side of the system may be constructed in the manner of a simple sheath system. In this respect, the inventive system offers some resemblance to those described in U.S. Pat. Nos. 6,280,465 and 6,833,003, the disclosures of which are herein incorporated by reference, or others. Alternatively, the stent restraint member(s) may be actuated by an internal pull wire or core wire. In such instances, exemplary proximal-side device construction approaches are provided in U.S. Pat. No. 6,736,839 and application Ser. Nos. 10/792,657, 10/792,679 and 10/792,684, filed on Mar. 2, 2004, and Ser. No. 10/991,721 filed Nov. 18, 2004, the disclosures of which are herein incorporated by reference.

In any case, the delivery guide preferably comprises a flexible atraumatic distal tip 106 of one variety or another. On the other end of the delivery device, a handle 110 may be provided. A body 112 of the handle may include one or more of a lever or slider 114 or other means (such as a trigger, knob or wheel) for actuating optional sheath/restraint or core member withdrawal. The delivery device handle may include a lock 116 to prevent inadvertent actuation. Similarly, handle 110 may include various safety or stop features and/or ratchet or clutch mechanisms to ensure one-way actuation.

Furthermore, a removable interface member 118 may be provided to facilitate taking the handle off of the delivery system proximal end 120. The interface may be lockable with respect to the body and preferably includes internal features for disengaging the handle from the delivery guide. Once accomplished, it will be possible to attach or “dock” a secondary length of wire 122 on the delivery system proximal end, allowing the combination to serve as an “exchange length” guidewire, thereby facilitating changing-out the balloon catheter or performing another procedure. Alternatively, a core member within the system may be an exchange-length wire.

FIG. 5 also shows packaging 150 containing at least one coiled-up delivery system 100. Packaging may include one or more of an outer box 152 and one or more inner trays 154, 156 with peel-away coverings as is customary in medical device product packaging. Naturally, instructions for use 158 may also be provided. Such instructions may be printed product included within packaging 150 or be provided in connection with another readable (including computer-readable) medium. The instructions may include provision for basic operation of the subject devices and associated methodology.

In support of such use, it is to be understood that various radiopaque markers or, features may be employed in the system to 1) locate the position of the stent, filter, or other component carried by the delivery system, 2) indicated device actuation and stent delivery and/or 3) locate the distal end of the corewire, guidewire or of any component carried thereon. As such, various platinum (or other radiopaque material) bands or other markers (such as tantalum plugs) may be incorporated into the system. Especially where the stent employed may shorten somewhat upon deployment, it may also be desired to align radiopaque features with the expected location (relative to the body of the guide member) of the stent upon deployment. For such purposes, radiopaque features may be set upon the core member of the delivery device proximal and distal of the stent.

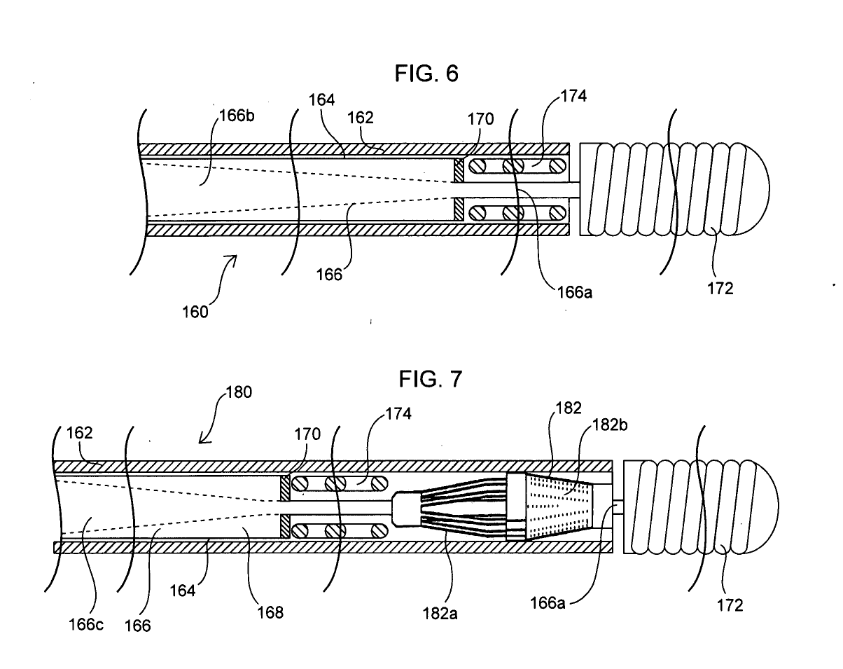

A feature of the present invention is that the inner member, i.e., the corewire/cladding combination, is configured to maximize the often conflicting objectives of flexibility and pushability. Turning now to FIGS. 6, 7 and 8, these show exemplary embodiments of stent delivery systems of the subject invention which achieve these two objectives.

Stent delivery system 160 of FIG. 6 includes an outer sheath or sleeve 162 and an inner member 164. Inner member 164 includes a corewire 166 that may have a constant diameter distal section 166a upon which a stent 172 is held in a constrained configuration by outer sheath 162. Proximal to stent 172, corewire 166 has a tapered section 166b with a continuously increasing diameter in the proximal direction. In other variations, the diameter of tapered section 166b may become smaller in a uniform taper or in varying tapers, for example, in steps. In either case, tapered section 166b may be an intermediate or transitional section between distal section 166a and a constant diameter proximal section 166c. Tapered section 166b increases flexibility of the corewire, particularly at a distal portion, without the introduction of stress raiser that introduce non-homogeneous performance and concentration of stress which can result in kinking.

Positioned over at least the intermediate section 166b is a cladding 168 made of a material having a modulus of elasticity lower than that of the corewire material which it, in a sense, replaces in the substantially constant diameter envelope defined by the inner member proximal of stop section 170, thereby imparting relatively increased flexibility to the inner member. Cladding 168 is shown having a constant outer diameter and an internal surface with a profile which matches or compliments that of corewire 166. In the illustrated embodiment, the profile of the inner surface of cladding 168 is tapered reversely from the taper of corewire 166 and, thus, substantially matches the profile of corewire 166.

The “match” in profiles between the outer surface of corewire 166 and the interior surface of cladding 168 need not be exact (but can be) and, in fact, may intentionally provide some space between the two where the gaps(s) may be filled with an adhesive material or other gap-filling material. Selective may be employed to fine-tune the flexibility along the length of the inner wire. The modulus of elasticity of the collective parts of the inner member generally becomes progressively lower distally along its length, selective spacing between the corewire and the cladding may be used at particular points along the length which interrupts the continuous decrease in the modulus of elasticity. The configuration of the gaps or spacing may be uniform or irregular, varying in volume, shape, etc.

Still other approaches may be taken in constructing and/or tuning the inner member used in the delivery system. Of these, certain known guidewire designs as described in U.S. Pat. Nos. 5,069,226, 5,797,857, Re. 36,628 and U.S. Pat. No. 6,019,737 each incorporated herein by reference for such purposes, may be employed. While these references fail to teach truncating the length of the resin envelope employed to provide a bare distal portion of the core member to mount an implantable medical device such as a stent, such modification can be made according to the present invention, even though doing so would defeat originally intended utility of the referenced guide wires.

Referring again to FIG. 6, another potential advantage of the delivery systems of the present invention is that the substantially constant outer diameter of cladding 168 serves to occupy the radial space between the corewire 166 and external sheath 162 up to a point proximal of the stent (e.g., directly adjacent the stent or about a blocker's width away) thereby minimizing the gap (i.e., the empty spacing) between inner member 164 and outer sheath 162. With larger gaps, misalignment occurs in which components in tension are pulled into a minimum radius configuration and components in compression are pushed into a maximum-radius configuration upon attempted actuation for deployment4. This miss-match of introduces unwanted variables into a stent delivery procedure which, as noted by the assignee hereof, may cause forward thrust of the non-optimal delivery system tip. Thus, the constant diameter inner member 164 and the minimized gaping between outer sheath 162 and inner member 164 provided by cladding 168 serve, together, to maintain outer sheath 162 and inner member 164 in substantially coaxially alignment with each other throughout the delivery and deployment procedure.

Maintaining such a relationship, in turn, may also help to efficiently and effectively transfer the pushing and torquing forces applied at the proximal end of the inner member to its distal end. As such, the user will have more control in any navigation desirably undertaken with the delivery system. Additionally, the cladding provides sufficient wall thickness to offer an adequate interface to abut the stent when withdrawing the sheath or when pushing the stent out of the sheath. Still further, it improves the column strength of the inner member (as compared to a bare tapered wire section) thus avoiding problems of buckling that can self-lock or at least drive-up the frictional forces between sheath and core member. As such, device actuation and resultant actuation forces may be improved.

Optionally, a stent stop section or stent blocker 170 adapted to abut the proximal end of the stent is carried by, connected to or integrally formed with the distal end of cladding 168. The blocker may be in the form of a ground-in shoulder section or a ring, band, a marker, a disc, etc. connected to cladding 168. Alternatively, the bare distal end of cladding 168 may function as the stent blocker. In any case, a stent stop or blocker surface is generally provided to hold the stent stationary within or relative to the delivery device upon withdrawal of the restraint.

Turning now to FIG. 7, another stent delivery system 180 of the present invention is provided. System 180 has a similar construct to the delivery system of FIG. 6. However, it includes an optional filter component 182 on distal section 166a of corewire 166 proximal to coil tip 172 and as such, functions as a combination embolic filter and stent delivery system. In the illustrated embodiment, filter 182 comprises an expansion frame having a plurality of outwardly biased struts 182a attached distally to a mesh 182b. However, filter component 182 may have any suitable construct, many of which are known in the art, such as those disclosed in U.S. Pat. No. 6,027,520, incorporated herein by reference in its entirety. As such, filter component 182 may be self-expanding (as illustrated) and retained in a constrained condition by outer sheath 162 in a manner similar to the manner by which self-expanding stent 174 is constrained prior to deployment. Alternatively, filter 182 may have an active configuration driven by shape memory alloy effect.

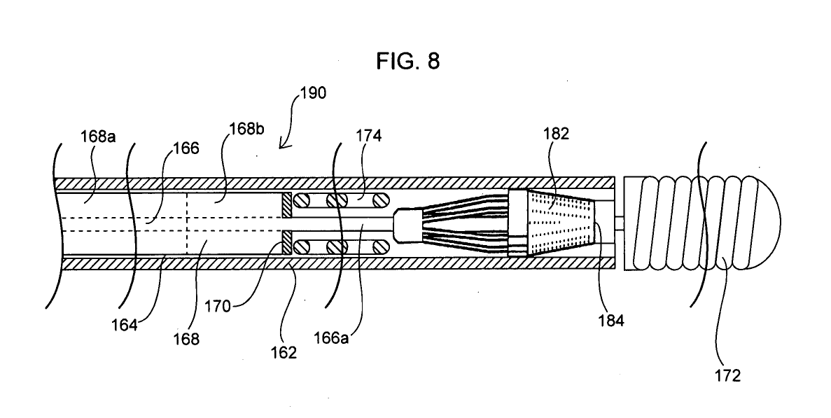

FIG. 8 illustrates yet another stent delivery system 190 of the present invention having an optional filter assembly 182 constructed and positioned similarly to that of the delivery system of FIG. 7. Here, however, a constant diameter corewire 166 is employed with a cladding 168 covering all but a distal section 166a of the corewire to form inner member 164. With a constant diameter, the flexibility of corewire itself is consistent along its length. Cladding 168 also has a constant outer diameter as well as a substantially constant inner diameter to match the profile of corewire 166. In order to vary the flexibility of the device to offer more guidewire like performance, the composition of cladding 168 is varyied in sections or continuously along its length. In one example, a proximal portion 168a of the cladding is composed of a composition having a higher modulus of elasticity than that of a distal section 168b.

Unlike the above described embodiments, the modulus of elasticity of at least at the proximal section 168a may be greater than that of corewire 166. However, the modulus of elasticity of at least a portion of 168b is most commonly less than that of corewire 166 as that is where most of the flexibility is required. The variance in flexibility of the cladding may be gradual and progressive or discrete and immediate. Upwards of three different flexibility/modulus or durometer sections may be employed in achieving desired device performance.

In the context of the angioplasty and stent deployment procedure described with respect to FIGS. 4E-4J, the use of deployment systems 170 and 180 are described as follows. With as the system serving in the capacity of delivery guide 80 in FIGS. 4E-4J, distal tip 172 and filter 182 are advanced distal of the lesion 62 and beyond the distal end of outer sheath 162. Either by self-expansion or blood flow within the artery, filter 182 is expanded (not illustrated) to operatively filter any emboli that may be released in the course of the predilatation procedure while allowing the filter blood to pass distally. The filter, then, remains deployed throughout the stent deployment and/or postdilatation procedures to capture any dislodged particulates. Stent 174 is deployed from deployment systems 170 and 180 in the same manner as described above with respect to delivery system 160 of FIG. 6. The distance between the stent and filter may vary. For a distal coronary application, however, the distance is typically between about 0.5 and about 5.0 mm.

The methods herein may be performed using the subject devices or by other means. The methods may all comprise the act of providing a suitable device. Such provision may be performed by the end user. In other words, “providing” (e.g., a delivery system) merely requires the end user to obtain, access, approach, position, set-up, activate, power-up or otherwise act to provide the requisite device in the subject method. Methods recited herein may be carried out in any order of the recited events which is logically possible, as well as in the recited order of events.

Though the invention has been described in reference to several examples, optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in the appended claims, the singular forms “a,” “an,” “said,” and “the” include plural referents unless the specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element—irrespective of whether a given number of elements are enumerated in the claim, or the addition of a feature could be regarded as transforming the nature of an element set forth n the claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of the claim language.

That being said, we claim:

1. A self-expanding stent delivery system comprising:
a sleeve for restraining a stent in a compressed form, an inner member for receipt within at least a portion of the sleeve, the inner member comprising a corewire and outer cladding,
the corewire comprising a distal stent-bearing section, and
the cladding covering at least a distal portion of the corewire proximal to the stent-bearing section,
wherein the inner member is adapted to provide increased flexibility in a proximal to distal fashion, and has a substantially constant diameter proximal to the stent bearing section.

2. The system of claim 1, wherein the stent-bearing section has a constant diameter.

3. The system of claim 1, wherein the corewire comprises a tapered section proximal to the distal stent-bearing section, and wherein the cladding covers at least the tapered section and has an inner surface having a space-filling profile that is complimentary to the tapered section of the corewire.

4. The system of claim 3, wherein the space-filling profile of the cladding comprises a taper having a slope inverse to that of the tapered section of the corewire.

5. The system of claim 3, wherein the corewire further comprises a constant diameter section proximal to the tapered section.

6. The system of claim 1, comprising a plurality of cladding sections with different material properties.

7. The system of claim 6, wherein the corewire comprises a constant diameter, and the plurality of sections have progressively lower modulus of elasticity in a proximal to distal direction.

8. The system of claim 1, wherein the cladding is made of material having a modulus of elasticity less than the corewire.

9. The system of claim 1, further comprising a stent.

10. The system of claim 9, wherein the stent comprises superelastic shape memory alloy material.

11. The system of claim 1, further comprising a filter operatively attached to the corewire at a location distal to the stent-bearing section.

12. The system of claim 11, wherein the filter comprises superelastic shape memory alloy material.

13. The system of claim 1, further comprising a stop for the stent at a distal end of the cladding.

14. The system of claim 13, wherein the stop is selected from one of an comprises one of a band and a disc.

15. A self-expanding stent delivery system comprising:
a corewire comprising a constant diameter distal section upon which the stent is received and a body section proximal to the distal section, and having a cladding covering at least the proximal section, wherein the cladding has a constant outer diameter;
a filter mechanism attached to the distal section of the corewire; and
an outer sleeve set over the corewire and cladding, wherein the outer sleeve is adapted to hold stent and filter in reduced states.

16. The system of claim 15, wherein the body section includes at least one tapered portion.

17. The system of claim 15, wherein the body section is constant in diameter.

18. The system of claim 15, wherein the filter mechanism is self-expanding.

19. The system of claim 15, wherein the filter mechanism is positioned distally of the stent.

20. The system of claim 15, further comprising a stop for the stent at a distal end of the cladding.
